(19) United States
(12) Patent Application Publication
(10) Pub. No.: US 2004/0157229 A1
(43) Pub. Date: Aug. 12, 2004

(54) Methods of profiling gene expression, protein or metabolite levels

(76) Inventors: Stephen John William Hole, Stein (CH); David Richard O'Reilly, Berkshire (GB)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/478,400

(22) PCT Filed: Jul. 17, 2002
(86) PCT No.: PCT/GB02/03243

(30) Foreign Application Priority Data
Jul. 19, 2001 (GB) 0117631.2

(51) Int. Cl. C12Q 1/68; G06F 19/00; G01N 33/48; G01N 33/50

(52) U.S. Cl. 435/6; 702/20

(57) ABSTRACT

The present invention relates to methods of analysing gene expression, metabolite or protein levels, the use of such methods to generate distinctive profiles of a cell, and the use of such profiles in the diagnosis of disease and mode of action of novel compounds used in the treatment of a cell. These simplified methods of obtaining molecular profiles of a cell have the advantage that they allow the skilled man to produce a profile of the levels of a chosen class of molecules in a cell, which may be used to distinguish between different treatments or different cellular states, whilst being produced from fewer data than are typically used in the production of existing profiles.

The present invention relates to methods of analysing gene expression, metabolite or protein levels, the use of such methods to generate distinctive profiles of a cell, and the use of such profiles in the diagnosis of disease and mode of action of novel compounds used in the treatment of a cell.

Recent technical advances have facilitated the contemporaneous measurement of large numbers of molecules in a cell. This has lead to the generation of complex profiles of specific classes of molecules, such as proteins, metabolites and mRNA, present in a cell. Typically, for each molecule in the chosen class of molecules present in the cell, a signal is generated, which correlates to the amount of that molecule present in the cell. Thus, a profile of gene expression in a cell is normally constructed from a vast number of individual measurements of mRNA levels of single genes. In other words, an expression profile is constructed from many signals, with each signal representing the level of expression of one gene. Similarly metabolite profiles are constructed with each signal representing the level of a single metabolite within the cell, and protein profiles are constructed with each signal representing the level of a single protein in the cell.

The construction of such profiles is extremely valuable in that it permits the detection of differences between, for example, different cellular states (e.g. between healthy and disease states) or, the effects of different treatments on a cell. However, such profiling experiments generate vast amounts of data, the sheer volume of which makes data storage, manipulation and interpretation highly problematic. Typically the individual datum points are then analysed and data may then be clustered for groups of molecules exhibiting for example, similar levels of expression or similar functional or structural characteristics in order to facilitate interpretation of the profile. However, this analysis stage means that a degree of knowledge (for example with respect to the level of one individual molecule in comparison to another individual molecule, or structural and/or functional knowledge of the indvidual molecules per se) is required prior to the generation of a user-friendly and readily interpretable profile.

The present invention addresses these difficulties and provides a greatly simplified method of obtaining molecular profiles of a cell, which has the advantage that it allows the skilled man to produce a profile of the levels of a chosen class of molecules in a cell, which may be used to distinguish between different treatments or different cellular states, whilst being produced from fewer data than are typically used in the production of existing profiles. Furthermore, the current invention provides a method of obtaining a useful molecular profile of a cell without the requirement for any knowledge about the individual molecules that are contributing to the profile.

The methods of the invention are generically applicable, in that they may be applied to different classes of molecules within a cell, e.g. they may be used to obtain profiles of the levels of metabolites or proteins or expressed genes within a cell, and they may be applied to cells from any source organism.

According to the present invention there is provided a method of characterising a cell or the effect of a treatment on a cell, which comprises the following steps: a) obtaining a plurality of aggregate signals, wherein each aggregate signal is representative of a combination of at least two indicator signals and each indicator signal is indicative of the level of a molecule in a cell; and b) generating from the plurality of aggregate signals a profile, which is characteristic of the cell or of the treatment on the cell, characterised in that each aggregate signal is obtained without analysing the contribution made by any one of the indicator signals to that aggregate signal.

The current inventive method is particularly advantageous in that the production of an aggregate signal prior to any data analysis stage reduces the number of data points used to generate a profile and simplifies any subsequent data handling and analysis. Profiles generated according to the method of the invention may be obtained from, and be characteristic of, an individual cell, a group of cells, a tissue or a whole organism. Profiles of the invention may also relate to and be characteristic of the level of the chosen class of molecules within a specific sub-cellular fraction.

The method of the invention may be used to generate characteristic profiles from any suitable class of molecules present in a cell. For example, the method may be used to characterise a cell according to gene expression levels, metabolite levels or protein levels.

Aggregate signals, from which profiles are obtained, are representative of at least two indicator signals, with each indicator signal being indicative of the level of an individual molecule in the cell. An aggregate signal thus represents the levels of a group of molecules in the cell. The composition of the group of molecules is preferably chosen at random with respect to the structure or function of the individual molecules: it is not necessary, and indeed it is preferred, that indicator signals for metabolites, proteins or genes are not grouped according to the biological or physiological pathways with which they are associated. Similarly, such indicator signals do not have to be grouped according to family membership that is defined either by structural class or on the basis of homology of an entire gene/protein and it is thus further preferred that indicator signals are not so grouped

In a further aspect of the invention, the composition of the group of molecules is chosen at random with respect to the level of individual molecules in the cell, group of cells, tissue or organism from which the profile is to be generated i.e. it is not necessary that the individual molecules comprising the group have the same or similar expression levels or that they are present in the same or similar amounts. Thus in a preferred embodiment an aggregate signal is representative of a combination of at least two indicator signals, wherein a first indicator signal is indicative of a level or amount of a first molecule and a second indicator signal is indicative of a level or amount of a second molecule, and the level or amount of the first molecule is considerably different to the level or amount of the second molecule. By “considerably different” it is meant that if the levels of the first and second molecules were to be analysed using any appropriate statistical or cluster analysis procedure prior to the generation of an aggregate signal, then the two levels would be deemed to be dissimilar. As described above, each indicator signal is indicative of the level of an individual molecule in the cell. It will be appreciated by the skilled man that the level of an individual molecule may either be zero or greater than zero.

Indicator signals are produced and detected by any appropriate means; radio-ligand detection, fluorescence detection, immunoassay, and enzyme-based assay all comprise examples of signal detection systems that may be employed in the method of the invention. Indicator signals may be converted to electronic signals to facilitate the generation of aggregate signals and subsequent profiles, however, it will be appreciated by the skilled man that such a conversion does not require knowledge of the comparative contribution of any individual molecule to any aggregate signal.

Indicator signals comprising an aggregate signal may be produced in close physical proximity to each other, thus facilitating production of the aggregate signal. Alternatively, aggregate signals may be obtained by randomly clustering indicator signals.

Where the method of the invention generates a gene expression profile, the indicator signals from which an aggregate signal is produced are indicative of the level of mRNA of genes expressed in the cell. Levels of mRNA may be measured by any suitable means, including for example nucleic acid hybridisation, quantitative PCR and any other means familiar to the skilled man.

In one embodiment, mRNA derived from a cell (or cDNA derived from cellular mRNA) is hybridised to a population of polynucleotides. Each of the polynucleotides corresponds to at least one gene capable of being expressed in the cell and each hybridisation event produces a hybridisation signal (i.e. an indicator signal) indicative of the level of expression of the gene or genes corresponding to the hybridised polynucleotide. Aggregate expression signals, which are representative of a combination of hybridisation signals produced from a sub-set of the population of polynucleotides, are then obtained without analysing the contribution made by any one of the hybridisation signals to that aggregate expression signal and used to generate an expression profile.

Any routine method that is well known in the art may be used to prepare mRNA and/or cDNA for use in the invention. It will also be appreciated by the skilled man that cellular mRNA for use in the invention may be in the form of a mixture of total cellular RNA, or it may be employed in a purified form, for example, it may be polyA purified.

The population of polynucleotides, to which mRNA or cDNA is hybridised, corresponds to genes that are capable of being expressed in the cell, since it is not as yet possible to predict how many or which genes will be expressed in a cell at a particular time or under a particular set of conditions. The entire population of polynucleotides may correspond to all of the genes capable of being expressed in the cell, including predicted gene sequences, genes of unknown function and genes for which a function has been assigned. Alternatively the population of polynucleotides may correspond to a sub-set of genes capable of being expressed in the cell (for example, only those genes for which a function has been prescribed, and/or known to be capable of being expressed under a specified set of conditions, or a proportion thereof).

Each individual member of the population of polynucleotides, to which mRNA or cDNA may be hybridised, corresponds to at least one gene capable of being expressed in the cell. By “corresponds to” it is meant that the polynucleotide may specifically hybridise to the mRNA transcript (or cDNA derived therefrom) of a gene capable of being expressed in the cell, if that gene is expressed in the cell. Thus a population of polynucleotides for use in the invention may comprise full- or partial-length genomic or cDNA clones, or polynucleotides derived therefrom, including synthetic nucleic acid sequences.

A member of the population of polynucleotides may correspond to a single gene, or it may correspond to more than one gene. Where a polynucleotide corresponds to a single gene, the sequence of the polynucleotide generally will be such that it is complementary to a nucleic acid (mRNA or cDNA) of a single gene. Where a polynucleotide corresponds to more than one gene, the sequence of the polynucleotide may be such that it is complementary to the nucleic acids of several (i.e. 2 or more) genes.

Each sub-set of polynucleotides from which an aggregate expression signal is obtained, corresponds to at least two genes capable of being expressed in the cell. Preferably a sub-set will correspond to any number of genes between 2 and 500, inclusive. In specific embodiments, a sub-set corresponds to 41, 166, 664 or 2656 genes.

It will be apparent to the skilled man that a sub-set may comprise a single member of the population of polynucleotides if that single member corresponds to more than one gene. Alternatively, a sub-set may comprise more than one polynucleotide, and each polynucleotide within such a sub-set may correspond to one or more genes capable of being expressed in the cell. It is thus possible to have sub-sets where the number of polynucleotides within the sub-set is either the same as, or different to, the number of genes to which that sub-set corresponds.

Polynucleotide populations for use in the invention may be in solution, or alternatively they may be bound to a solid support. Suitable solid supports may be in the form of a planar surface, e.g. a membrane, including nylon and PVDF membranes, or a glass slide. Where a planar surface is employed as the solid support, the population of polynucleotides may form an array of discrete spots on the planar surface. Each discrete spot will comprise at least one member of the population of polynucleotides and will thus correspond to at least one gene capable of being expressed in the cell. In a preferred aspect, each discrete spot will comprise a sub-set of the population of polynucleotides from which an aggregate expression signal is obtained.

As an alternative, the population of polynucleotides may be bound, either directly or indirectly, to a plurality of beads. Where beads are employed as the solid support, it is preferred that each bead is bound to a sub-set of the population of polynucleotides from which an aggregate expression signal is obtained. It is also preferable that each bead is uniquely identifiable, for example, by each bead being associated with a fluorescent label.

In a particularly preferred embodiment, all polynucleotide members of the population will comprise a polyT region and a random sequence of nucleotides. Hybridisation of mRNA to such a population results in the binding of different numbers of mRNA molecules to the different members of the population. This particular embodiment provides an example of how indicator signals may be grouped together at random: in this case grouping is based on the sequence at the 5′ end of the gene and not on gene function or homology over the entire length of the gene. Another important feature of this embodiment is that an aggregate expression signal can be obtained from an individual member of the population of polynucleotides, i.e. a single polynucleotide acts as the subset of polynucleotides from which an aggregate expression signal is obtained.

It is further preferred that such individual polynucleotide members can be readily distinguished from each other. This may be achieved, for example, by binding individual polynucleotide members to separate beads, or arraying them in spots on a planar membrane such that each discrete spot is comprised of an individual polynucleotide member.

By altering the length of the random sequence of nucleotides, the number of mRNA molecules binding to an individual polynucleotide member can be altered, since the probability of a greater number of mRNA molecules hybridising to an individual polynucleotide will increase as the length of random sequence in the polynucleotide decreases.

In further embodiments the method of the invention may be used to generate a metabolite profile of a cell, or a protein profile of a cell.

Aggregate signals as described herein form yet a further aspect of the invention, for example, in one embodiment there is provided an aggregate signal for use in generating a profile that is characteristic of a cell or the effect of a treatment on a cell, wherein the aggregate signal is representative of a combination of at least two indicator signals and each indicator signal is indicative of the level of a molecule in the cell and wherein the aggregate signal is obtained without analysing the contribution made by any one of the indicator signals to that aggregate signal. In a second embodiment there is provided an aggregate signal that is representative of a random combination of at least two indicator signals. In a third embodiment there is provided an aggregate signal that is representative of a random combination at least two indicator signals and the aggregate signal is obtained without analysing the contribution made by any one of the indicator signals to that aggregate signal.

The invention also extends to a profile of the level of the chosen class of molecules in a cell, the profile comprising a plurality of aggregate signals of the invention.

Where the profile is a gene expression profile, each aggregate signal is indicative of the aggregate expression level of a sub-set of genes, and each sub-set comprises at least two genes. Similarly, a protein profile will comprise a plurality aggregate signals wherein each aggregate signal is indicative of the aggregate level of at least two proteins present in the cell, and a metabolite profile will comprise a plurality of aggregate signals wherein each aggregate signal is indicative of the aggregate level of at least two metabolites present in the cell.

Profiles according to the invention can be used to correlate the level of the chosen class of molecules (mRNA, protein or metabolite) with a particular cellular state. The term “state” as applied herein to a cell, can refer to a physiological state of the cell, which may result from environmental stress, disease, or treatment with an exogenous agent, or it can refer to a developmental state of the cell. Comparisons between profiles obtained from cells in two different states permits the generation of a profile that may be used to characterise either state, and is characteristic of both. For direct comparison between such profiles, it will be appreciated that the composition of molecules in the sub-sets from which aggregate signals are generated will be the same in each of the profiles compared. In general, comparisons will be made between profiles obtained from cells in a test state (e.g. from cells in a diseased state or treated with an exogenous agent) and profiles obtained from cells in a control state (e.g. from cells in a healthy state or cells that have not been treated with the exogenous agent).

Gene expression profiles are particularly useful in diagnosing a specific cellular state, for example in diagnosing disease where the disease causes an alteration in number and/or the level of genes expressed. Comparisons between the gene expression profile for a diseased cell and that of a healthy or control cell will permit the generation of a profile that is characteristic of that disease. A panel of profiles may thus be constructed with each profile being characteristic of a particular disease state. Where it is suspected that cells may be diseased, their profile, in comparison to that of healthy cells, may be obtained and reviewed alongside a profile known to be characteristic of a specific disease. In this way, disease may be diagnosed.

Gene expression profiles may be used in a similar manner to verify or identify the way in which a particular treatment (for example, treatment with a compound) affects a cell. The mode of action of a compound may be verified or identified by comparing the profile obtained from cells treated with the compound to a profile obtained from untreated or appropriate control cells. This profile may then be reviewed alongside profiles that have been obtained for chemicals having known modes of action and the mode of action of the test compound may then be verified or identified.

The invention also extends to novel chemicals, the mode of action of which has been verified or identified using any of the profiles described herein.

Various aspects and embodiments of the present invention will now be illustrated in more detail by way of example. It will be appreciated that modification of detail may be made with out departing from the scope of the invention.

### BRIEF DESCRIPTION OF THE FIGURES

FIG. 1 Dendogram showing relatedness of treatments when expression of individual genes is analysed.

FIG. 2 Dendogram showing relatedness of treatments when expression is analysed for groups of 2 genes.

FIG. 3 Dendogram showing relatedness of treatments when expression is analysed for groups of 10 genes.

FIG. 4 Dendogram showing relatedness of treatments when expression is analysed for groups of 41 genes.

FIG. 5 Dendogram showing relatedness of treatments when expression is analysed for groups of 166 genes.

FIG. 6 Dendogram showing relatedness of treatments when expression is analysed for groups of 664 genes.

FIG. 7 Dendogram showing relatedness of treatments when expression is analysed for groups of 2656 genes.

### EXAMPLE

Production of Gene Expression Profiles of Plant Cells Using Aggregate Expression Signals

Plants were treated with ALS (0.5 ppm), PDS (50 ppm), Sterol (150 ppm) and AOZ (0.5 ppm), and harvested 3 days after treatment. RNA was isolated from treated plants, as well as control plants that had received no treatment, using standard procedures.

A total of 18 hybridisation experiments were carried out using Arabidopsis Gene Expression Microarrays or GEMs (Incyte), each comprising polynucleotides that correspond to 7968 different genes (i.e. a subset of the total number of Arabidopsis genes). Each GEM was hybridised with two RNA samples: either, a “treated” sample and a “control” sample or with two different control RNA samples. A summary of the hybridisation experiments carried out is given in Table 1.

TABLE 1
Summary of GEM hybridisations. Controls were either carried
out at the same time as (Cb controls), or independently
of (Ca controls), the treatments.

| Experiment | Sample 1 | Sample 2 |
|---|---|---|
| 1 | ALS rep 1 | Control Cb1 |
| 2 | AOZ rep 1 | Control Ca1 |
| 3 | Control Ca1 | Control Ca1 |
| 4 | ALS rep 1 | Control Ca1 |
| 5 | Sterol rep 1 | Control Cb1 |
| 6 | PDS rep 2 | Control Cb1 |
| 7 | Control Ca1 | Control Ca1 |
| 8 | PDS rep 2 | Control Ca1 |
| 9 | Control Ca1 | Control Cb1 |
| 10 | Control Cb1 | Control Cb1 |
| 11 | PDS rep 1 | Control Cb1 |
| 12 | ALS rep 2 | Control Cb1 |
| 13 | ALS rep 2 | Control Ca1 |
| 14 | Control Ca1 | Control Cb1 |
| 15 | Control Cb1 | Control Cb1 |
| 16 | Control Cb1 | Control Cb4 |
| 17 | PDS rep 1 | Control Ca1 |
| 18 | Sterol rep 2 | Control Cb1 |

Expression Profiles Obtained from Individual Hybridisation Signals

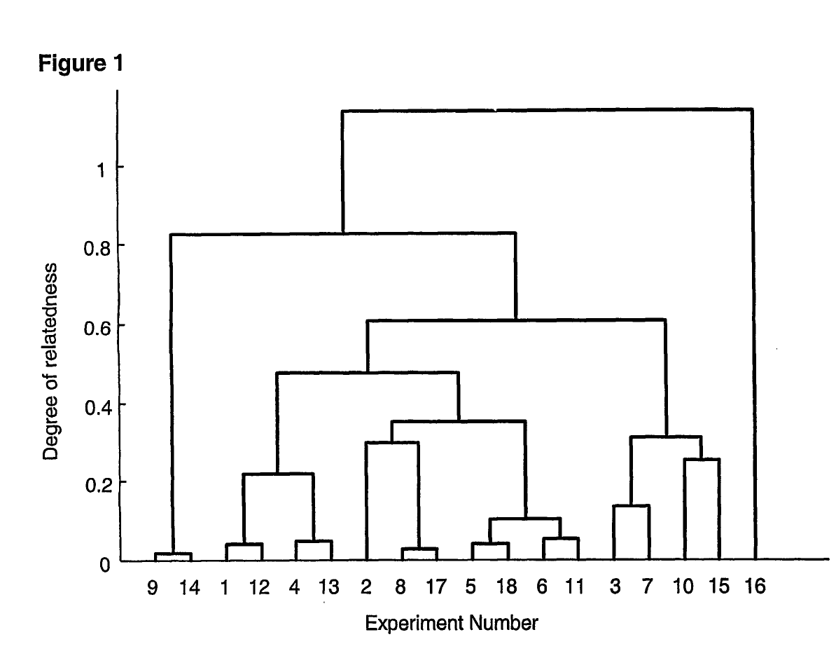

The signals from each GEM were normalised according to the total signal. The log of the ratio of treatment vs. control was calculated for each gene. A distance algorithm was then used to cluster the experiments according to similarity of gene expression ratios. The dendogram obtained (FIG. 1) is based on 7968 genes and represents gene expression profiles produced from individual genes (i.e. 7968 groups of 1 gene each).

This analysis clearly separated all the treatments from all the controls. However, some degree of control bias was observed, with experiments appearing to cluster according to the control, rather than according to the treatment.

Expression Profiles Obtained from Aggregate Expression Signals

The effect of considering aggregate expression levels of various sized groups of genes on the clustering of treatments was then examined. The group sizes were chosen on the assumption that, in practise, the evaluation of aggregate expression levels would be done with oligo-dTN₍ₙ₎ primers. If n=1, there are 3 different possible primers (oligo dT(G/A/C)), n=2 gives 12 different primers (oligo dT(G/A/C)(G/A/T/C)) etc. Genes were put into groups randomly as follows; each gene was assigned a number at random, the genes were then listed by this number and assigned to groups according to their position in the list. For the purpose of this in silico analysis the group size was kept constant, however, in practise the group size is likely to vary for different primers. As a result, some of the genes (those at the end of the list) were left out of the analysis. Table 2 shows the different group sizes analysed, and the number of genes omitted in each case.

TABLE 2
Profiles were generated from aggregate expression
signals representing different number of genes. The
in silico analysis was performed on the basis that in
practise, aggregate expression signals would be
generated from oligo-dTN₍ₙ₎ primers

| | No of aggregate expression | No of Genes per | |
|---|---|---|---|
| n | signals | Group | No of Genes left out |
| 1 | 3 | 2656 | |
| 2 | 12 | 664 | |
| 3 | 48 | 166 | |
| 4 | 192 | 41 | 96 |
| 5 | 768 | 10 | 288 |
| 6 | 3072 | 2 | 1824 |

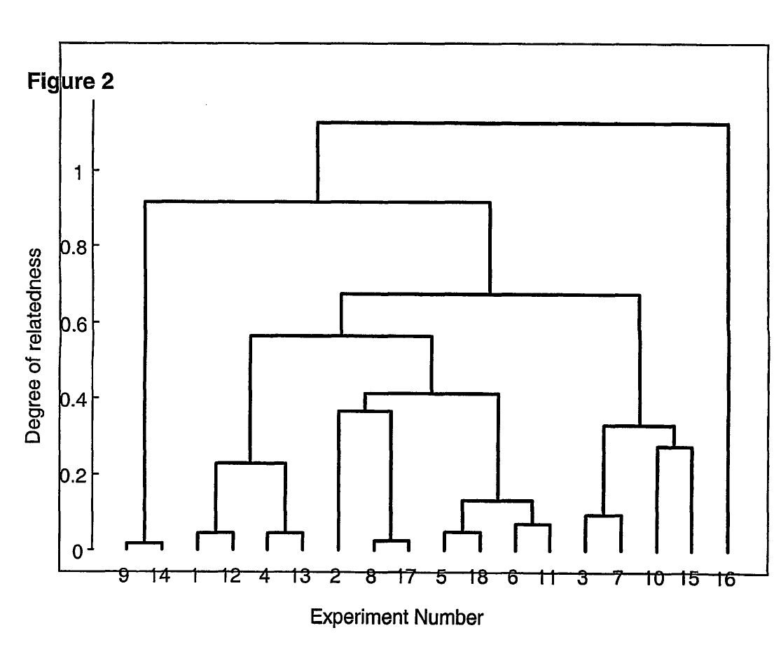

For each group of genes, the signals of the constituent genes were summed. The signals were then normalised, log ratios calculated, and the treatments clustered as described above. The dendograms obtained are shown in FIGS. 2 to 7.

Results

In the analysis of individual genes (FIG. 1), the ALS treated samples clustered together well, apart from the other treatments. The PDS and Sterol treatments are less clearly resolved, with the controls appearing to exert more influence over the pattern of clustering than the treatments. However, PDS and Sterol treatments might be expected to give similar expression profiles.

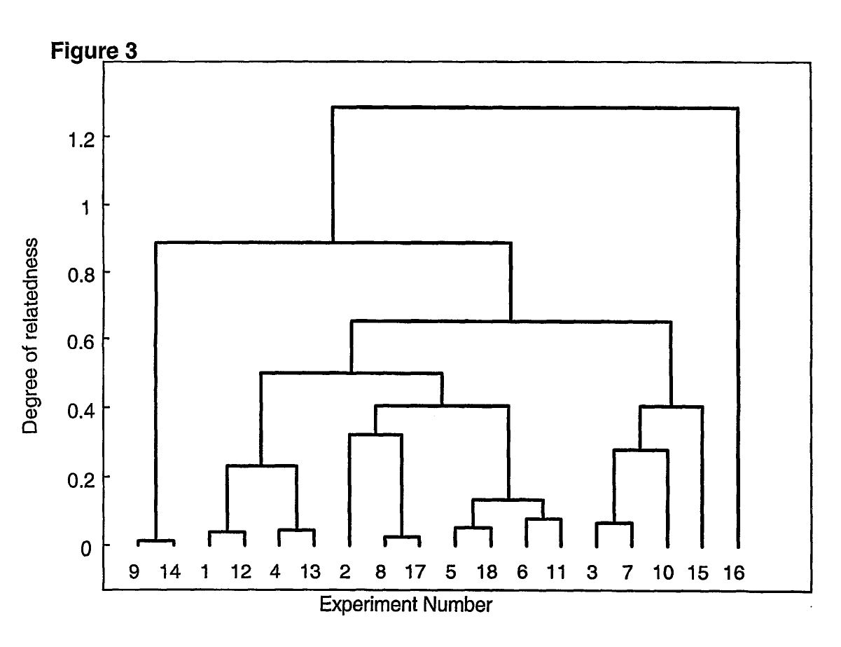

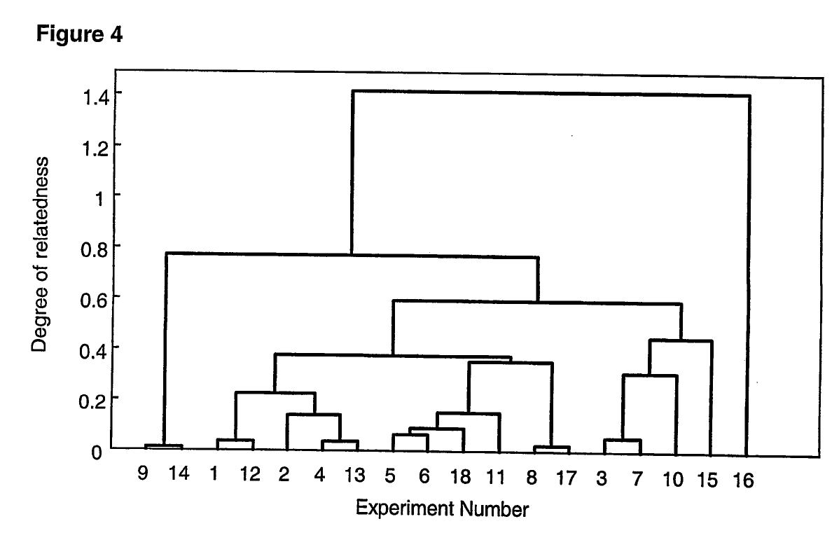

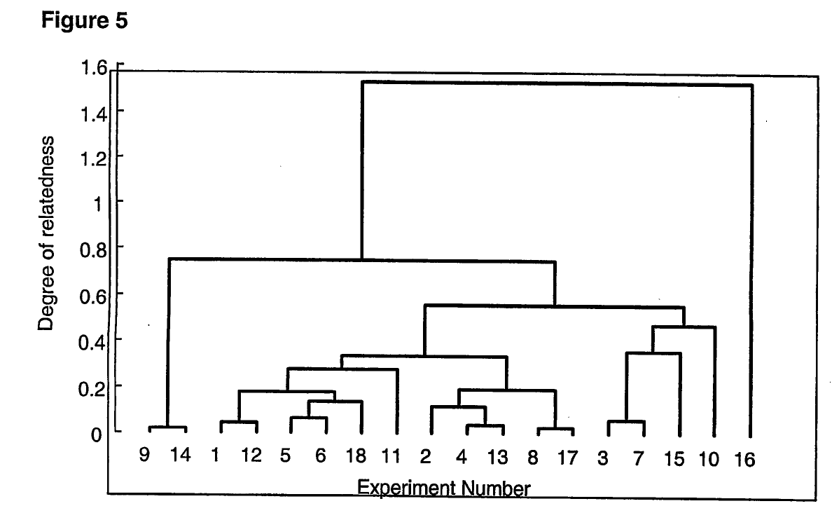

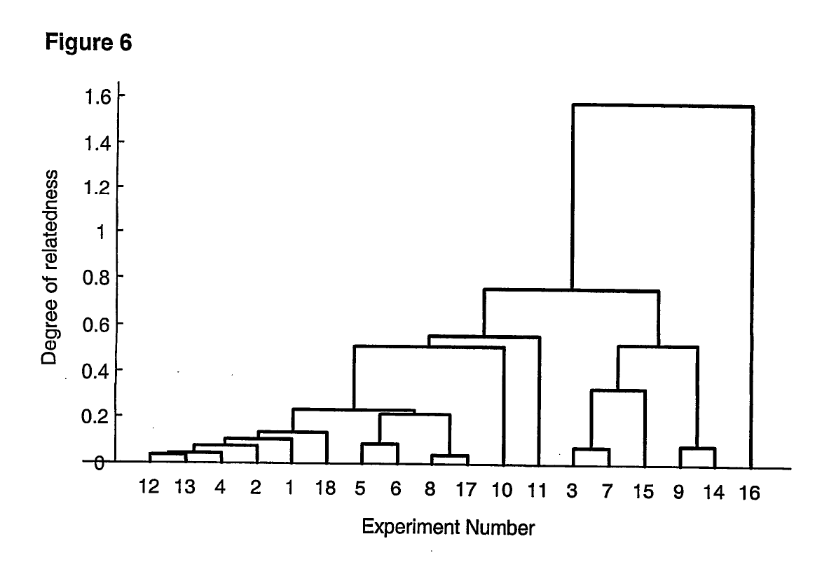

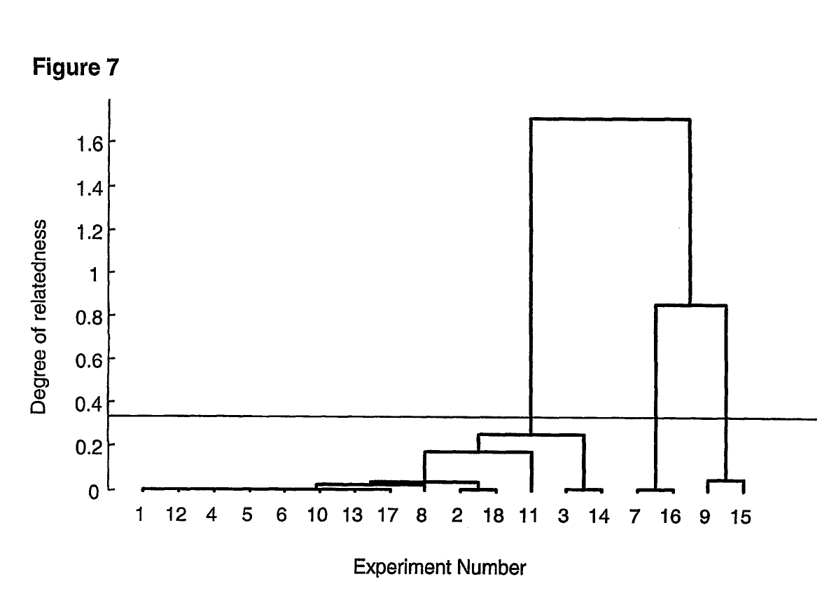

Essentially the same clustering pattern was generated when the samples were analysed as 3072 groups of two genes each (FIG. 2) or 768 groups of 10 genes each (FIG. 3). When the data were analysed as 192 groups of 42 genes each (FIG. 4), the ALS treatments were still well separated from the Sterol and PDS treatments. However, the resolution among the PDS and Sterol treatments has degraded slightly. When the data were aggregated into 48 groups of 166 genes (FIG. 5), the signal due to the different controls predominated over signals from the individual treatments. However, if one only considers samples that were profiled against Cb1 controls (i.e. controls carried out at same time as treatments), the ALS inhibitors still cluster together, apart from the other treatments, and all treatments are clearly separated from all controls. Only when the data were analysed as 12 groups of 664 genes each (FIG. 6) do some of the controls begin to group with the treatments. Poor resolution is obtained when the data were analysed as 3 groups of 2565 genes (FIG. 7).

Conclusions

Analysis of the aggregate expression of groups of genes allows clustering of treatments according to mode of action of the treatment. No knowledge of the individual gene identities is required.

Essentially no change was observed in the clustering pattern with 768 groups of 10 genes. It was thus possible to reduce the number of data points to be analysed 10-fold without affecting the clustering. Treatments that typically cluster well could still be resolved with 48 groups of 166 genes.

1. A method of characterising a cell or the effect of a treatment on a cell, which comprises the following steps:
a) obtaining a plurality of aggregate signals, wherein each aggregate signal is representative of a combination of at least two indicator signals and each indicator signal is indicative of the level of a molecule in a cell;
b) generating from the plurality of aggregate signals a profile that is characteristic of the cell or of the treatment on the cell;
characterised in that each aggregate signal is obtained without analysing the contribution made by any one of the indicator signals to that aggregate signal

2. A method according to claim 1, wherein each indicator signal is indicative of the level of an mRNA molecule in the cell.

3. A method according to claim 1, wherein each indicator signal is indicative of the level of a metabolite in the cell.

4. A method according to claim 1, wherein each indicator signal is indicative of the level of a protein in the cell.

5. A method of characterising a cell or the effect of a treatment on a cell, which comprises the following steps:
a) hybridising mRNA derived from a cell, or cDNA derived from cellular mRNA, to a population of polynucleotides, wherein each polynucleotide corresponds to at least one gene capable of being expressed in the cell and each hybridisation event produces a hybridisation signal indicative of the level of expression of the gene or genes corresponding to the hybridised polynucleotide;
b) obtaining a plurality of aggregate expression signals, wherein each aggregate expression signal is representative of a combination of hybridisation signals produced from a sub-set of the population of polynucleotides and each sub-set corresponds to at least two genes capable of being expressed in the cell, and wherein each aggregate expression signal is obtained without analysing the contribution made by any one of the hybridisation signals to that aggregate expression signal;
c) generating from the plurality of aggregate expression signals an expression profile, which is characteristic of the cell or characteristic of the treatment of the cell.

6. A method according to claim 5, wherein a sub-set corresponds to between 2 and 500 (inclusive) randomly selected genes capable of being expressed in the cell.

7. A method according to claim 5 or claim 6, wherein a sub-set corresponds to 41, 166, 664, or 2656 randomly selected genes capable of being expressed in the cell.

8. A method according to any one of the previous claims, wherein the number of genes to which a first sub-set corresponds is different from the number of genes to which a second sub-set corresponds.

9. A method according to any one of the previous claims wherein the number of polynucleotides comprising a sub-set is the same as the number of genes to which that sub-set corresponds.

10. A method according to any one of claims 5 to 8 wherein the number of polynucleotides comprising a sub-set is less than the number of genes to which that sub-set corresponds.

11. A method according to any one of claims 5 to 10, wherein the population of polynucleotides is bound to a solid support.

12. A method according to claim 11, wherein the solid support is in the form of a planar surface and the population of polynucleotides forms an array of discrete spots on the planar surface.

13. A method according to claim 12, wherein the discrete spots each comprise a sub-set from which an aggregate expression signal is obtained.

14. A method according to claim 11, wherein the solid support is in the form of a plurality of beads and each bead is uniquely identifiable.

15. A method according to claim 14, wherein each bead is bound to a sub-set of the population of polynucleotides from which an aggregate expression signal is obtained.

16. A profile of gene expression levels in a cell, the profile comprising a plurality of aggregate signals, wherein each aggregate signal is representative of a combination of at least two indicator signals and each indicator signal is indicative of the level of expression of a gene in the cell, and wherein the aggregate signal is obtained without analysing the contribution made by any one of the indicator signals to that aggregate signal

17. A gene expression profile obtained by comparing first and second profiles, each according to claim 16, wherein the first profile is obtained from a cell in a first state, and the second profile is obtained from a cell in a second state, and the composition of genes in the sub-sets used in each profile is the same.

18. A gene expression profile according to claim 17, wherein the first state results from a first treatment and the second state results from a second treatment.

19. A gene expression profile according to claim 17 or claim 18 wherein the first state is a test state and the second state is a control state.

20. A gene expression profile according to claim 19, wherein the test state is a potentially diseased state.

21. Use of a gene expression profile according to claim 20, to diagnose a diseased state.

22. A gene expression profile according to claim 19, wherein cells in the first state have been treated with a compound of known mode of action.

23. A gene expression profile according to claim 19, wherein cells in the first state have been treated with a compound of unknown mode of action.

24. Use of a gene expression profile according to claim 22 or claim 23, to identify or verify the mode of action of a test compound.

25. A novel compound, the mode of action of which has been identified or verified through the use of a gene expression profile according to claim 22 or claim 23.
